(19) United States
(12) Patent Application Publication
(10) Pub. No.: US 2006/0111343 A1
(43) Pub. Date: May 25, 2006

(54) Oxcarbazepine dosage forms

(75) Inventors: Anandi Krishnan, Vashi (IN); Muthaiyyan Esakki Kannan, Kopar Khairane (IN); Atul Vishvanath Patil, Airoli (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/264,836

(22) Filed: Nov. 1, 2005

Related U.S. Application Data
(60) Provisional application No. 60/623,926, filed on Nov. 1, 2004.

(51) Int. Cl. A61K 31/55; A61K 31/165

(52) U.S. Cl. 514/217; 514/617

(57) ABSTRACT

Dosage forms of oxcarbazepine or a pharmaceutically acceptable salt thereof and a pH modifying agent for use in oral administration are provided.

### PRIORITY

This application claims the benefit under 35 U.S.C. §119 to Provisional Application No. 60/623,926, filed No. 1, 2004 and entitled “OXCARBAZEPINE IMMEDIATE RELEASE TABLETS”, the contents of which are incorporated by reference herein.

### BACKGROUND OF THE INVENTION

1. Technical Field

The present invention relates generally to dosage forms containing oxcarbazepine or a pharmaceutically acceptable salt thereof as the active pharmaceutical ingredient and a process for preparing same.

2. Description of the Related Art

Oxcarbazepine, 10,11-dihydro-10-oxo-5H-dibenzo[b,]azepine-5-carboxamide, is a widely used antiepileptic drug having poor solubility in water. Drug insolubility is one of the major challenges in the development of many pharmaceutical products. Attempts have been made to enhance the dissolution rate and therefore the availability/bioavailability of oxcarbazepine by reducing the particle size of the pure oxcarbazepine to a size of 2 to 12 μm. The particle size reduction typically requires special equipment such as an air-jet mill. The process also involves a long time and a loss of the material during the process. Furthermore, the micronized particles are difficult to handle during the manufacturing of the formulation. Some of the other earlier attempts to enhance the dissolution rate involved the use of a wetting agent in the formulation. See, e.g., PCT Application No. WO 02/094774 A2. The use of a wetting agent in the formulation may show enhanced dissolution rate in the in-vitro conditions, but the in-vitro in-vivo correlation has not been established.

Accordingly, there is a clear need to provide alternative dosage forms of oxcarbazepine which can be prepared using less complicated and expensive processes and fulfill all prerequisites for pharmaceutical use, e.g., sufficient solubility of the active substance in water.

### SUMMARY OF THE INVENTION

In accordance with one embodiment of the present invention, an oral dosage composition is provided comprising a prophilactically or therapeutically effective amount of oxcarbazepine or a pharmaceutically acceptable salt thereof and a pH modifier comprising an amide of the general formula R¹R²NH wherein R¹ is a C₁-C₁₈ hydrocarbon and R² is a C₁-C₁₈ hydrocarbon containing two or more hydroxyl groups.

In accordance with a second embodiment of the present invention, a solid oral dosage composition comprising a prophilactically or therapeutically effective amount of an active pharmaceutical ingredient comprising oxcarbazepine or a pharmaceutically acceptable salt thereof and a pH modifier comprising an amide of the general formula R¹R²NH wherein R¹ is a C₁-C₁₈ hydrocarbon and R² is a C₁-C₁₈ hydrocarbon containing two or more hydroxyl groups.

In accordance with a third embodiment of the present invention, a solid oral dosage composition comprising a prophilactically or therapeutically effective amount of an active pharmaceutical ingredient comprising oxcarbazepine or a pharmaceutically acceptable salt thereof and a pH modifier comprising an amide of the general formula R²R²NH wherein R¹ is a C₁-C₁₈ hydrocarbon and R² is a C₁-C₁₈ hydrocarbon containing two or more hydroxyl groups, wherein the dosage form is an immediate release dosage form.

In accordance with a fourth embodiment of the present invention, a pharmaceutical composition is provided comprising a prophilactically or therapeutically effective amount of an active pharmaceutical ingredient comprising oxcarbazepine or a pharmaceutically acceptable salt thereof having a particle size D₉₀ of not less than about 50 μm and a pH modifier comprising an amide of the general formula R¹R²NH wherein R¹ is a C₁-C₁₈ hydrocarbon and R² is a C₁-C₁₈ hydrocarbon containing two or more hydroxyl groups.

In accordance with a fifth embodiment of the present invention, a pharmaceutical composition is provided comprising a prophilactically or therapeutically effective amount of an active pharmaceutical ingredient comprising oxcarbazepine or a pharmaceutically acceptable salt thereof having a particle size D₉₀ of about 80 to about 140 μm and a pH modifier comprising an amide of the general formula R¹R²NH wherein R¹ is a C₁C₁₈ hydrocarbon and R² is a C₁-C₁₈ hydrocarbon containing two or more hydroxyl groups.

According to another aspect of the present invention a process for the preparation of oxcarbazepine dosage forms for oral administration is provided, the process comprising the steps of (a) treating oxcarbazepine alone or a blend of oxcarbazepine and one or more pharmaceutical acceptable excipients with a pH modifier comprising an amide of the general formula R¹R²NH wherein R¹ is a C₁-C₁₈ hydrocarbon and R² is a C₁-C₁₈ hydrocarbon containing two or more hydroxyl groups; and (b) formulating into suitable dosage form.

The present invention provides oxcarbazepine dosage forms which can be prepared by a simple, less time consuming and economical process.

### DEFINITIONS

Unless defined otherwise, all technical and scientific terms used herein have the same meaning as commonly understood by one of ordinary skill in the art to which this invention belongs. Although any method and material similar or equivalent to those described herein can be used in the practice or testing of the present invention, the preferred methods and materials are described.

Unless stated to the contrary, any use of the words such as “including”, “containing”, “comprising”, “having” and the like, means “including without limitation” and shall not be construed to limit any general statement that it the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention is defined by the appended claims.

The expression “solid oral dosage composition” as used herein shall be understood to mean all solid oral dosage forms including, but not limited to, powders, tablets, dispersible granules, capsules, caplets, sachets and the like.

The term “therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

The term “delivering” as used herein means providing a therapeutically effective amount of an active ingredient to a particular location within a host means causing a therapeutically effective blood concentration of the active ingredient at the particular location.

The term “pharmaceutically acceptable” as used herein means that which is useful in preparing a pharmaceutical composition that is generally non-toxic and is not biologically undesirable and includes that which is acceptable for veterinary use and/or human pharmaceutical use.

The term “subject” or “a patient” or “a host” as used herein refers to mammalian animals, preferably human.

The term “excipients” as used herein means a component of a pharmaceutical product that is not an active ingredient such as, for example, fillers, diluents, carriers and the like. The excipients that are useful in preparing a pharmaceutical composition are preferably generally safe, non-toxic and neither biologically nor otherwise undesirable, and are acceptable for veterinary use as well as human pharmaceutical use.

The term “adsorbant” as used herein means an agent whose surface area is larger to the irregularity and roughness of the surface. The active chemical entity gets bound on this surface by forming Van der Waals interactions, and hydrogen bonding forces of attraction (forms physical bond) and thus the drug surface is completely covered by the adsorbing material, in turn helps in masking the bitter taste of the pharmaceutically active entity.

The term “sweetener” as used herein means an agent who imparts the artificial sweetening to the formulation to help in masking the unacceptable taste for the dosage form. Useful sweeteners include, but are not limited to, sodium saccharine, dextrose, sucrose, aspartame, magnasweet and the like and mixtures thereof.

The term “disintegrating agent” as used herein means an agent which in contact with water swells and fragments the solid dosage form. Useful disintegrating agents include, but are not limited to, croscarmellose sodium, crospovidone, and the like and mixtures thereof. In the present invention, crospovidone may be used as a disintegrating agent, which swells in the presence of water and helps in fragmenting the tablet dosage form. Additional exemplary disintegrants include, by way of example and without limitation, starches, e.g., sodium starch glycollate, corn starch, potato starch, pre-gelatinixed and modified starched thereof, clays, e.g., bentonite, microcrystallinc cellulose (e.g. Avicel™), carsium (e.g. Amberlite™), alginates, sodium starch glycolate, gums, e,g., agar, guar, locust bean, karaya, pectin, tragacanth, combinations thereof and other such materials known to those of ordinary skill in the art.

The term “effervescing agent” as used herein means such materials known to those of ordinary skill in the art. Here citric acid is used in the dry mix intragranularly and sodium bicarbonate (dried) is used extra granularly. These ingredients in the presence of water undergo a chemical reaction to evolve carbon dioxide by forming effervescence.

In the present embodiment the flavorings agents can be used intragranularly and extragranulerly. The flavorings agents are chemical moieties such as esters and aldehydes which imparts the flavoring effect to the dosage forms.

The term “antioxidant” as used herein means an agent which inhibits oxidation and is thus used to prevent the deterioration of preparations by the oxidative process. Such compounds include, by way of example and without limitation, ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisultfite and other such materials known to those of ordinary skill in the art.

The term “buffering agent” as used herein means a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “binders” as used herein means substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include, but are not limited to, starch, poly(ethylene glycol), guar gum, polysaccharide, bentonites, sugars, invert sugars, poloxamers (PLURONIC™ F68, PLURONIC™ f127), collagen, albumin, celluloses in nonaqueous solvents, combinations therof and the like. Other binders include, for example, poly(propylene glycol), polyoxycthylene-polypropylene copolymer, polycthylene ester, polyethylene sorbitan ester, poly(ethylene oxide), microcrystalline cellulose, poly(vinylpyrrolidone), combinations thereof and other such materials known to those of ordinary skill in the art.

The terms “diluent” or “filler” as used herein means inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein means agents used in tablet and capsule formulations to improve flow-properties during tablet compression and to produce an anti-caking caking effect. Such compounds include, by way of example and without limitation, colloidal silica, calcium silicate, magnesium silicate, silicon hydrogel, cornstarch, talc, combinations thereof and other such materials known to those of ordinary skill in the art.

The terms “lubricant” or “lubricating agent” as used herein means substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### DETAILED DESCRIPTION OF THE PREFERRED EMBODIMENTS

One embodiment of the present invention relates to formulations containing the active pharmaceutical ingredient oxcarbazepine or a pharmaceutically acceptable salt thereof, and a pH modifying agent. In another embodiment, the formulations of the present invention contain the active pharmaceutical ingredient oxcarbazepine or a pharmaceutically acceptable salt thereof having a particle size D₉₀ of not less than about 50 μm and a pH modifying agent. In yet another embodiment of the present invention, the formulations of the present invention contain the active pharmaceutical ingredient oxcarbazepine or a pharmaceutically acceptable salt thereof having a particle size D₉₀ of about 80 to about 140 μm, which can be achieved using conventional techniques, and a pH modifying agent.

The prophylactic or therapeutic dose of oxcarbazepine or pharmaceutically acceptable salt thereof can vary widely depending upon a variety of factors including, for example, the body weight, general health, sex, diet, time and route of administration, rates of absorption and excretion, combination with other drugs, the severity of the particular condition being treated, etc. Generally, the amount of oxcarbazepine or a pharmaceutically acceptable salt thereof may range from about 20 w/w % to about 90 w/w % of the dosage form.

The pH modifier for use in the formulations of the present invention include at least an amide as represented by the general formula R¹R²NH wherein R¹ is a linear or branched C₁-C₁₈ hydrocarbon and R² is a linear or branched C₁-C₁₈ hydrocarbon containing two or more hydroxyl groups. R¹ can be, for example, a linear or branched C₁-C₁₈ alkyl group and preferably a linear or branched C1-C₆ alkyl group, a substituted or unsubstituted C₄-C₁₈ alicyclic or an alkylalicyclic radical or a a substituted or unsubstituted C₅-C₁₈ alkylaryl where the alkyl group is from about 1 to about 6 carbon atoms. R² can be, for example, a linear or branched C₁-C₁₈ alkyl group and preferably a linear or branched C₄-C₁₀ alkyl group, a substituted or unsubstituted C₄-C₁₈ alicyclic or an alkylalicyclic radical or a a substituted or unsubstituted C₅-C₁₈ alkylaryl where the alkyl group is from about 1 to about 6 carbon atoms, with R² further containing at least two hydroxyl groups, preferably at least four hydroxyl groups and preferably at least six hydroxyl groups. In another embodiment, R² can contain from four to ten hydroxyl groups. Examples of such pH modifiers for use herein include meglumine and the like.

The pH modifier should generally be used in an amount which is sufficient to provide an alkaline medium for solubilisation of the drug. This amount may vary with the type of pH modifier used. Generally, the pH modifier can be present in an amount ranging from about 0.10 w/w % to about 5 w/w % of the dosage form and preferably from about 1 w/w % to about 3 w/w % of the dosage form.

The formulations of the present invention can also contain one or more pharmaceutically acceptable excipients. Examples of such excipients include, but are not limited to, diluents, binders, disintegrants, lubricants, glidants, coloring agents, flavoring agents and sweeteners, and the like and mixtures thereof which are chemically and physically compatible with oxcarbazepine.

Suitable diluents for use in the dosage forms of the present invention may be any such pharmaceutically acceptable excipient which give bulk to the oxcarbazepine composition such as those discussed hereinabove. Examples of such diluents include, but are not limited to, starch, microcrystalline cellulose, lactose, glucose, mannitol, alginates, alkali earth metal salts, clays, polyethylene glycols and the like and mixtures thereof. Generally, the diluent may be present in the dosage forms of the present invention in an amount ranging about 10 w/w to about 80 w/w % of the dosage form.

Suitable binders for use in the dosage forms of the present invention may be any such pharmaceutically acceptable excipient which have cohesive properties to act as binders such as those discussed hereinabove. Examples of such binders include, but are not limited to, starch, highly dispersed silica, mannitol, lactose, polyethylene glycol, cross-linked polyvinylpyrrolidone, cross-linked carboxymethyl cellulose, hydroxypropyl methyl cellulose, hydroxy propyl cellulose and the like and mixtures thereof. Generally, the binder may be present in the dosage forms of the present invention in an amount ranging about 0.10 w/w % to about 10 w/w % of the dosage form.

Suitable disintegrants for use in the dosage forms of the present invention include, but are not limited to, starches or modified starches such as sodium starch glycolate, corn starch, potato starch or pregelatinized starch, clays such as bentonite, montmorillonite or veegum; celluloses such as microcrystalline cellulose, hydroxypropyl cellulose or carboxymethyl cellulose, algins such as sodium alginate or alginic acid; cross-linked cellulose such as croscarmellose sodium, gums such as guar gum or xanthan gum; cross-linked polymers such as crospovidone; effervescent agent such as sodium bicarbonate and citric acid; and the like and mixtures thereof. Generally, the disintegrant may be present in the dosage forms of the present invention in an amount ranging about 0.10 w/w % to about 10 w/w % of the dosage form.

Suitable lubricants for use in the dosage forms of the present invention include, but are not limited to, talc, magnesium stearate, other alkali earth metal stearate like calcium, zinc etc., lauryl sulphate, hydrogenated vegetable oil, sodium benzoate, sodium stearyl tumarate, glyceryl monostearate, PEG 4000 and the like and mixtures thereof. Generally, the lubricant may be present in the dosage forms of the present invention in an amount ranging about 0.10 w/w % to about 5 w/w % of the dosage form.

Suitable glidants for use in the dosage forms of the present invention include, but are not limited to, colloidal silicon dioxide, talc and the like and mixtures thereof. Generally, the glidant may be present in the dosage forms of the present invention in an amount ranging about 0.10 w/w % to about 5 w/w % of the dosage form.

Suitable coloring agent for use in the dosage forms of the present invention may be any colorant used in pharmaceuticals which is approved and certified by the FDA. Useful colorants include, but are not limited to, Lake of Tartrazine, Lake of Quinoline Yellow, Lake of Sunset Yellow and Lake of Erythrosine, Lack of Carmosine Ponceau, Allura Red. Preferably coloring agents for the present invention are Lake of Tartrazine and Lake of Quinoline Yellow, as these are comparatively cheaper and may give excellent uniformity of color to the dosage form.

The desired dosage forms of the present invention can be any suitable dosage form. In one embodiment, the desired dosage form is a solid dosage form, e.g., powders, tablets, dispersible granules, capsules, caplets, sachets and the like. A preferred oral solid dosage form is a tablet. Tablets according to the present invention may be produced by any standard tabletting technique, e.g. by wet granulation, dry granulation or direct compression. The tablets can be of any size and shape. Also, the dosage forms of the present invention may be in the form of film-coated immediate release tablets. The coating composition may be a rapidly disintegrating composition which may include one or more film forming agents, plasticizers, anti tacking agent, fillers, channel forming agent, coloring agent, opacifiers, flavoring agents, sweeteners and the like and mixtures thereof. In one embodiment, the dosage forms are provided as immediate release dosage forms, e.g., immediate release tablets, capsules and the like.

In one embodiment, a process for preparing the dosage form of the present invention involves the dilution of oxcarbazepine or pharmaceutically acceptable salt thereof with one or more diluent/filler(s), blending with the filler, roll compaction of the blend, milling of the blend in, for example, a comminuting mill, followed by granulation in high shear mixers. The granules are dried and reduced to the desired size, blended with one or more disintegrants and then lubricated with one or more lubricants. The lubricated blend can then be compressed to tablets. If desired, the tablets can be coated with a rapidly disintegrating coating composition.

The following examples are provided to enable one skilled in the art to practice the invention and are merely illustrative of the invention. The examples should not be read as limiting the scope of the invention as defined in the claims.

### EXAMPLES 1-3 AND COMPARATIVE EXAMPLES A AND B

The percent w/w formula compositions of Examples 1-3 (within the scope of the present invention) and Comparative Examples A and B (outside the scope of the present invention) are set forth in Table 1.

TABLE 1

| | Example 1 | Example 2 | Example 3 | Comp. Ex. A | Comp. Ex. A |
|---|---|---|---|---|---|
| Ingredient | (% w/w) | (% w/w) | (% w/w) | (% w/w) | (% w/w) |
| Oxcarbazepine | 75.00 | 75.00 | 75.00 | 75.00 | 75.00 |
| Microcrystalline cellulose (Avicel PH 101) | 14.90 | 14.15 | 14.40 | 18.00 | 18.00 |
| Hydroxypropyl methyl cellulose (Methocel E-3 LV) | 2.10 | 2.10 | 2.10 | 3.00 | 3.00 |
| Meglumine | 2.00 | 3.00 | 2.00 | — | — |
| Crospovidone (Polyplasdone XL 10) | 5.00 | 5.00 | 5.00 | 3.00 | 3.00 |
| Colloidal silicon dioxide (Aerosil 200) | 0.40 | 0.40 | 0.40 | — | — |
| Magnesium stearate | 1.10 | 1.10 | 1.10 | 0.50 | 0.50 |
| Water | q.s | q.s | q.s | q.s | q.s |

The detailed stepwise procedure for the manufacturing process is as follows.

1. Oxcarbazepine was blended with Avicel PH 101 in a blender.

2. The blend of step 1 was compacted in a roll compactor to get compacts/slugs.

3. The compacts/slugs of step 2 were milled in a comminuting mill using a 0.25 mm screen.

4. The milled mass of step 3 was blended in a high shear mixer.

5. The blended mass of step 4 was granulated in the mixer using a solution of Methocel E-3 LV 1.

6. The granules of step 5 were dried.

7. The dried granules of step 6 were reduced to the desired size in a comminuting mill using a 1.50 mm screen.

8. The sized granules of step 7 were blended with the crospovidone and Aerosil 200.

9. The blended granules of step 8 were lubricated with magnesium stearate.

10. The lubricated granules of step 9 were compressed into tablets.

11. The compressed tablets of step 10 were coated using a rapidly disintegrating coating composition (Opadry from M/S Colorcon).

In Examples 1 and 2, meglumine was added prior to the compaction stage. In Example 3, meglumine was added during the granulation stage along with the binder fluid. In Comparative Example B, the oxcarbazepine was milled using a ball mill in order to reduce the particle size which is simple cost effective equipment.

The oxcarbazepine tablets of Examples 1-3 and Comparative Examples A and B were tested in a dissolution media of 900 ml of 0.75% sodium lauryl sulfate in water at 37° C. using a USP Dissolution Apparatus Type II at an agitation of 60 RPM. For a further comparison, Trileptale tablets 600 mg (containing colloidal silicon dioxide, crospovidone, hydroxypropyl methyl cellulose, magnesium stearate, microcrystalline cellulose, polyethylene glycol, talc, titanium dioxide and yellow iron oxide as excipients) of Novartis was also tested under the same conditions for the comparative study. The data obtained from the dissolution tests are set forth in Tables 2 and 3. The dissolved oxcarbazepine is expressed in cumulative percent of drug dissolved over an elapsed time period in minutes.

TABLE 2
Dissolution profile of oxcarbazepine tablets of Comparative Examples
A and B in comparison with Trileptal ® tablets

| Oxcarbazepine | | | | | | |
|---|---|---|---|---|---|---|
| tablets | 5 min. | 10 min. | 15 min. | 30 min. | 45 min. | 60 min. |
| Comp. Ex. A* | 22 | 40 | 49 | 62 | 70 | 74 |
| Comp. Ex. B* | 15 | 32 | 46 | 61 | 69 | 73 |
| Trileptal ® | 39 | 74 | 83 | 90 | 91 | 94 |

*Uncoated Tablets

TABLE 3
Dissolution profile of oxcarbazepine tablets of Examples
1-3 in comparison with Trileptal ® tablets

| Tablet | | | | | | |
|---|---|---|---|---|---|---|
| Samples | 5 min. | 10 min. | 15 min. | 30 min. | 45 min. | 60 min. |
| Example 1* | 52 | 60 | 70 | 80 | 86 | 89 |
| Example 2 | 38 | 63 | 75 | 86 | 93 | 94 |
| Example 3 | 41 | 69 | 75 | 85 | 90 | 93 |
| Trileptal | 39 | 74 | 83 | 90 | 91 | 94 |

*Uncoated Tablets

The dissolution data of Tables 2 and 3 clearly shows that oxcarbazepine tablets formed in the absence of a pH modifier such as meglumine of Comparative Examples A and B (outside the scope of the present invention) did not give the desired dissolution profile when prepared using the unmicronized oxcarbazepine API as compared to the oxcarbazepine tablets formed with unmicronized oxcarbazepine API and a pH modifying agent of Examples 1-3 (within the scope of the present invention). Accordingly, the inclusion of the pH modifying agent in the formulation advantageously avoids the micronization of oxcarbazepine API, which requires special equipments and strict controls.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

What is claimed is:

1. An oral dosage composition comprising a prophilactically or therapeutically effective amount of oxcarbazepine or a pharmaceutically acceptable salt thereof and a pH modifier comprising an amide of the general formula R¹R²NH wherein R¹ is a C₁-C₁₈ hydrocarbon and R² is a C₁-C₁₈ hydrocarbon containing two or more hydroxyl groups.

2. The oral dosage composition of claim 1, wherein the oxcarbazepine or pharmaceutically acceptable salt thereof has a median particle size of not less than about 50 μm.

4. The oral dosage composition of claim 1, wherein the oxcarbazepine or pharmaceutically acceptable salt thereof has a median particle size of about 80 to about 140 μm.

5. The oral dosage composition of claim 1, wherein R¹ is a straight or branched C₁-C₁₈ alkyl group.

6. The oral dosage composition of claim 1, wherein R² is a straight or branched C₁-C₁₈ alkyl group containing two to ten hydroxyl groups.

7. The oral dosage composition of claim 1, wherein R¹ is a straight or branched C₁-C₁₈ alkyl group and R² is a straight or branched C₄-C₁₀ alkyl group containing two to ten hydroxyl groups.

8. The oral dosage composition of claim 1, wherein the amide is meglumine.

9. The oral dosage composition of claim 1, wherein the pH modifier is present in an amount of about 0.10 w/w % to about 5 w/w % of the dosage form.

10. The oral dosage composition of claim 1, wherein the oxcarbazepine or pharmaceutically acceptable salt thereof has a median particle size of not less than about 50 Am and in the amide R¹ is a straight or branched C₁-C₁₈ alkyl group and R² is a straight or branched C₄-C₁₀ alkyl group containing two to ten hydroxyl groups.

11. The oral dosage composition of claim 1, wherein the oxcarbazepine or pharmaceutically acceptable salt thereof has a median particle size of about 80 to about 140 μm and in the amide R¹ is a straight or branched C₁-C₁₈ alkyl group and R² is a straight or branched C₄-C₁₀ alkyl group containing two to ten hydroxyl groups.

12. The oral dosage composition of claim 1, wherein the oxcarbazepine or pharmaceutically acceptable salt thereof has a median particle size of not less than about 50 μm and the pH modifier is meglumine.

13. The oral dosage composition of claim 1, wherein the oxcarbazepine or pharmaceutically acceptable salt thereof has a median particle size of about 80 to about 140 μm and the pH modifier is meglumine.

14. The oral dosage composition of claim 1, which is an oral solid dosage composition.

15. The oral dosage composition of claim 1, in the form of a tablet.

16. The oral dosage composition of claim 15, wherein the tablet is coated.

17. The oral dosage composition of claim 14, wherein the oral solid dosage composition is an immediate release oral solid dosage composition.

18. The oral dosage composition of claim 12, in the form of a tablet.

19. The oral dosage composition of claim 18, wherein the tablet is coated.

20. The oral dosage composition of claim 1, further comprising one or more pharmaceutically acceptable excipients.

21. The oral dosage composition of claim 20, wherein the one or more pharmaceutically acceptable excipients is selected from the group consisting of a diluent, binder, disintegrant, lubricant, glidant, coloring agent, flavoring agent, sweetener and mixtures thereof.

22. A tablet comprising oxcarbazepine or a pharmaceutically acceptable salt thereof having a median particle size of not less than about 50 μm and a pH modifying agent comprising an amide of the general formula R¹R²NH wherein R¹ is a C₁-C₁₈ hydrocarbon and R² is a C₁-C₁₈ hydrocarbon containing two or more hydroxyl groups.

23. The tablet of claim 22, wherein the amide is meglumine.

24. The tablet of claim 22, containing a hydrophilic permeable outer coating thereon.

25. A tablet comprising oxcarbazepine or a pharmaceutically acceptable salt thereof having a median particle size of about 80 to about 140 μm and a pH modifying agent comprising an amide of the general formula R¹R²NH wherein R¹ is a C₁-C₁₈ hydrocarbon and R² is a C₁-C₁₈ hydrocarbon containing two or more hydroxyl groups.

26. The tablet of claim 25, wherein the amide is meglumine.

27. The tablet of claim 25, containing a hydrophilic permeable outer coating thereon.

28. A process for the preparation of an oxcarbazepine dosage form for oral administration, the process comprising the steps of (a) treating oxcarbazepine or a pharmaceutically acceptable salt thereof alone or a blend of oxcarbazepine and one or more pharmaceutical acceptable excipients with a pH modifier comprising an amide of the general formula R¹R²NH wherein R¹ is a C₁-C₁₈ hydrocarbon and R² is a C₁-C₁₈ hydrocarbon containing two or more hydroxyl groups; and (b) formulating into a suitable dosage form.

29. The process of claim 28, wherein the oxcarbazepine or pharmaceutically acceptable salt thereof has a median particle size of not less than about 50 μm and in the amide R¹ is a straight or branched C₁-C₁₈ alkyl group and R² is a straight or branched C₁-C₁₈ alkyl group containing two to ten hydroxyl groups.

30. The process of claim 28, wherein the oxcarbazepine or pharmaceutically acceptable salt thereof has a median particle size of about 80 to about 140 μm and in the amide R¹ is a straight or branched C₁-C₁₈ alkyl group and R² is a straight or branched C₁-C₁₈ alkyl group containing two to ten hydroxyl groups.

31. The process of claim 28, wherein the oxcarbazepine or pharmaceutically acceptable salt thereof has a median particle size of not less than about 50 μm and the pH modifier is meglumine.

32. The process of claim 28, wherein the oxcarbazepine or pharmaceutically acceptable salt thereof has a median particle size of about 80 to about 140 μm and the pH modifier is meglumine.

33. The process of claim 28, wherein the dosage form is an oral solid dosage composition.

34. The process of claim 28, wherein the dosage form is a tablet.

35. The process of claim 34, wherein the tablet contains a coating thereon.
